(19) United States
(12) Patent Application Publication
(10) Pub. No.: US 2008/0058278 A1
(43) Pub. Date: Mar. 6, 2008

(54) ATTENUATION OF HYPEROXIA-INDUCED CELL DEATH WITH MITOCHONDRIAL ALDEHYDE DEHYDROGENASE

(75) Inventors: Dong Xu, Kansas City, MO (US); William Truog, Kansas City, MO (US)

Correspondence Address: ERICKSON & KLEYPAS, L.L.C., 800 W. 47TH STREET, SUITE 401, KANSAS CITY, MO, 64112, US

(73) Assignee: THE CHILDREN'S MERCY HOSPITAL, Kansas City, MO (US)

(21) Appl. No.: 11/764,761

(22) Filed: Jun. 18, 2007

Related U.S. Application Data
(60) Provisional application No. 60/814,270, filed on Jun. 16, 2006.

(51) Int. Cl. A61K 31/70; A61P 11/00; C12N 15/00; C12N 15/87; C12N 5/06

(52) U.S. Cl. 514/44; 435/320.1; 435/325; 435/371; 435/375; 435/455

(57) ABSTRACT

Oxygen toxicity is one of the major risk factors in the development of the chronic lung disease or bronchopulmonary dysplasia in premature infants. Using proteomic analysis, we discovered mitochondrial aldehyde dehydrogenase (mtALDH or ALDH2) was down-regulated in neonatal rat lung after hyperoxic exposure. To study the role of mtALDH in hyperoxic lung injury, we overexpressed mtALDH in human lung epithelial cells (A549) and found that mtALDH significantly reduced hyperoxia-induced cell death. Compared to control cells (Neo-A549), the necrotic cell death in mtALDH overexpressing cells (mtALDH-A549) decreased from 25.3% to 6.5%, 50.5% to 9.1% and 52.4% to 15.06% after 24-, 48- and 72-hour hyperoxic exposure, respectively. The levels of intracellular and mitochondria-derived reactive oxygen species (ROS) in mtALDH-A549 cells after hyperoxic exposure were significantly lowered compared to Neo-A549 cells. mtALDH overexpression significantly stimulated extracellular signal regulated kinase (ERK) phosphorylation under normoxic and hyperoxic conditions. Inhibition of ERK phosphorylation partially eliminated the protective effect of mtALDH in hyperoxia-induced cell death, suggesting ERK activation by mtALDH conferred cellular resistance to hyperoxia. mtALDH overexpression augmented Akt phosphorylation and maintained the total Akt level in mtALDH-A549 cells under normoxic and hyperoxic conditions. Inhibition of PI3K activation by LY294002 in mtALDH-A549 cells significantly increased necrotic cell death after hyperoxic exposure, indicating that PI3K/Akt activation by mtALDH played an important role in cell survival after hyperoxia. Taken together, these data demonstrate that mtALDH overexpression attenuates hyperoxia-induced cell death in lung epithelial cells through reduction of ROS, activation of ERK/MAPK and PI3K/Akt cell survival signaling pathways.

### RELATED APPLICATION

This application claims the priority benefit of U.S. provisional patent application Ser. No. 60/814,270, filed on Jun. 16, 2006. The teachings and content of that application are hereby expressly incorporated by reference herein.

### SEQUENCE LISTING

This application contains a sequence listing in both paper format and in electronic format filed through the electronic filing system. The sequence listing on paper is identical to the sequence listing on electronic format, and all are expressly incorporated by reference herein.

### BACKGROUND OF THE INVENTION

1. Field of the Invention

The present invention is concerned with the amelioration, reduction, or prevention of oxygen toxicity. More particularly, the present invention is concerned with the amelioration, reduction, or prevention of cell injury and/or death resulting from oxygen toxicity. Still more particularly, the present invention is concerned with the prevention, reduction in the incidence of or likelihood of an individual developing chronic lung disease or bronchopulmonary dysplasia as a result of being exposed to toxic levels of oxygen. Still more particularly, the present invention is concerned with the activation of pathways that eliminate or reduce the generation of reactive oxygen species (ROS). Even more particularly, the present invention is concerned with the use of mitochondrial aldehyde dehydrogenase (mtALDH) for the amelioration, reduction, or prevention of cell injury and/or death resulting from oxygen toxicity and the generation of ROS, as well as the prevention, reduction in the incidence or likelihood of an individual developing chronic lung disease or bronchopulmonary dysplasia. Still more particularly, the present invention is concerned with the activation of the ERK/MAPK pathway and/or the activation of the Akt cell survival pathway. Even more particularly, the present invention is concerned with the use of mtALDH for the amelioration, reduction, or prevention of cell injury and/or death resulting from oxygen toxicity and the generation of ROS, as well as the prevention, reduction in the incidence of, or likelihood of, an individual developing chronic lung disease or bronchopulmonary dysplasia.

2. Description of the Prior Art

Patients including premature newborns with respiratory distress are frequently treated with supplemental oxygen. After the supplemental of oxygen therapy, some patients develop acute and chronic lung injury because of oxygen toxicity. Hyperoxic lung injury is characterized by pulmonary inflammation, hemorrhage and eventually cell death of pulmonary capillary endothelial cells and alveolar epithelia cells, which result in impaired gas exchange and pulmonary edema (5, 8). Currently there are no safe and known effective adjunctive treatments to be administered with supplemental oxygen to ameliorate or prevent oxygen induced epithelial cell injury and/or death.

Reactive oxygen species (ROS) generated during supplemental oxygen therapy are extremely cytotoxic and they have the ability to interact with and alter essential cell components, including proteins, lipids, carbohydrates and DNA (15, 36). Decreased antioxidant capacity of lung tissue during hyperoxia may contribute to the lung injury (14, 37). Thus, the elimination or reduction of excess ROS generation, either by blocking ROS formation or increasing antioxidant production, should result in reduced cellular oxidative injury with ultimate protection of cells from hyperoxia-induced cell death (3, 11).

Hyperoxia induces both apoptotic (6, 12) and nonapoptotic cell death in pulmonary epithelial cells (13, 26). Cell death is thought to be the major contributing factor in the development of acute or chronic lung injury after oxygen therapy. Apoptosis is a tightly regulated process. Hyperoxia induces apoptotic cell death in lung epithelial cells by activation of both intrinsic and extrinsic apoptosis pathways (23, 32). Non-apoptotic cell death, including necrosis and oncosis, is characterized by cell and organelle swelling, vacuolization, and increased membrane permeability (18, 21, 40). Hyperoxia primarily induces necrotic cell death in cultured A549 cells, a pulmonary type II epithelial cell line derived from human lung adenocarcinoma. A small portion of the cell death is due to apoptosis in cultured A549 cells after hyperoxia. Two cell survival signaling pathways, extracellular signal regulated kinase/mitogen activated protein kinase (ERK/MAPK) and phosphatidylinositol 3-kinase-Akt (PI3K/Akt), are implicated in the survival of pulmonary epithelial cells after hyperoxic exposure. Hyperoxia activate thes ERK/MAPK pathway and suppresses the PI3K/Akt pathway in lung epithelial cells (7, 10, 20, 35, 39). Increased ERK activation or constitutive expression of the active form of Akt delays hyperoxia-induced cell death and increases animal survival after prolonged hyperoxic exposure (7, 20).

Mitochondria are the major source of ROS production under normoxic or hyperoxic conditions (4). Mitochondrial aldehyde dehydrogenase (mtALDH or ALDH2) is a nuclear-encoded mitochondrial enzyme that is localized in mitochondrial matrix (25). The role of mtALDH in lung epithelial cells during oxidative stress or hyperoxia is not known. In this study, we found that mtALDH was down-regulated in the neonatal rat lung after hyperoxic exposure using proteomic analysis. Moreover, mtALDH overexpression in lung epithelial cells activated both ERK/MAPK and PI3K/Akt signaling pathways and protected lung epithelial cells from hyperoxia-induced cell death.

As is understood by those of skill in the art, the possibility of developing hyperoxic lung injury varies by individual and their tolerance of various levels of oxygen or resistance to ROS. For example, typical atmospheric oxygen concentrations and partial pressure of oxygen levels (both of which are referred to herein as “oxygen levels”) may be toxic to some premature infants, but not to the majority of the population. Additionally, the duration of exposure to oxygen levels is also related to the development of hyperoxic lung injury. At concentration levels that are at the lower end of toxic concentration levels, increased exposure time may increase the toxicity and/or effect of toxicity. Similarly, high concentration levels may be less toxic if exposure is only for a short duration.

### SUMMARY OF THE INVENTION

The present invention overcomes the deficiencies of the prior art and provides a distinct advance in the state of the art. In one aspect of the present invention, methods for ameliorating, reducing the incidence or severity of, or preventing injury and damage, up to and including death, to epithelial tissues resulting from oxygen toxicity are provided. Generally, the method includes using mtALDH. In more detail, the expression of mtALDH is enhanced in cells susceptible to damage from ROS. The present invention also provides methods for preventing or reducing the incidence of, severity of, or likelihood of an individual developing chronic lung disease or bronchopulmonary dysplasia as a result of being exposed to toxic levels of oxygen. Again, the method generally includes using mtALDH. In more detail, the expression of mtALDH is enhanced in cells susceptible to damage from ROS. Additionally, the present invention provides methods for activating pathways that eliminate or reduce the generation of reactive oxygen species (ROS). In general, the methods of the present invention use mitochondrial aldehyde dehydrogenase (mtALDH) to ameliorate, reduce, or prevent cell injury and/or death resulting from oxygen toxicity and the generation of ROS, as well as to prevent or reduce the incidence of or likelihood of an individual developing chronic lung disease or bronchopulmonary dysplasia.

In summary, mtALDH is down-regulated in the neonatal rat lung after prolonged hyperoxic exposure. Overexpression of mtALDH confers lung epithelial cell resistance to hyperoxia-induced cell injury and/or death. The cytoprotection of mtALDH in lung epithelial cell is mediated through ROS reduction, and activation of ERK/MAPK and PI3K/Art cell survival signaling pathways.

### BRIEF DESCRIPTION OF THE DRAWINGS

FIG. 1A is a photograph of a gel identifying mtALDH from an unknown and down-regulated protein from neonatal rat lung tissue exposed to normoxic conditions;

FIG. 1B is a photograph of a gel identifying mtALDH from an unknown and down-regulated protein from neonatal rat lung tissue exposed to hyperoxic conditions;

FIG. 1C is a graph depicting mtALDH activities in A549 cells under normoxic or hyperoxic conditions for 3 days (n=3, data were expressed as mean±SD) using isolated mitochondrial protein from attached cells for the mtALDH activity assay;

FIG. 2A is a photograph of a Western blot showing the increased presence of mtALDH in transfected cells, as compared with untransfected cells;

FIG. 2B is a photograph of the results of an immunofluorescent study comparing mtALDH-A549 cells with Neo-A549 cells;

FIG. 2C is a graph illustrating the total mtALDH activities in mtALDH-A549 and Neo-A549 cells;

FIG. 2D is another graph illustrating the total mtALDH activities in mtALDH-A549 and Neo-A549 cells;

FIG. 3A is a graph comparing necrotic cell death over 72 hours of normoxic exposure between mtALDH-A549 and Neo-A549 cells in a trypan blue exclusion assay;

FIG. 3B is a graph comparing necrotic cell death over 72 hours of hyperoxic exposure between mtALDH-A549 and Neo-A549 cells in a trypan blue exclusion assay;

FIG. 3C is a graph comparing apoptotic cell death over 48 hours of hyperoxic and normoxic exposure between mtALDH-A549 and Neo-A549 cells after Annexin V staining;

FIG. 4A is a graph comparing intracellular ROS levels as measured by flow cytometry after staining with H₂DCFA;

FIG. 4B is a graph comparing mitochondria-derived ROS levels as measured by flow cytometry after staining with dihydrorhodamine 123;

FIG. 5A is a photograph of a Western blot illustrating the stimulation of ERK phosphorylation in mtALDH-A549 and Neo-A549 cells by both mtALDH and hyperoxia over 72 hours of exposure to hyperoxic conditions;

FIG. 5B is a photograph of a Western blot illustrating ERK phosphorylation in mtALDH-A549 and Neo-A549 by both normoxia and hyperoxia over 48 hours;

FIG. 5C is a graph illustrating the quantified levels of phosphorylated ERK from FIG. 5B;

FIG. 6A is a graph illustrating necrotic cell death in U0126 pretreated or non-pretreated Neo-A549 and mtALDH-A549 cells after 48 hours of normoxic or hyperoxic exposure, as measured by a trypan blue exclusion assay;

FIG. 6B is a graph illustrating necrotic cell death in U0126 pretreated or non-pretreated Neo-A549 and mtALDH-A549 cells after 48 hours of normoxic or hyperoxic exposure, as measured by a lactate dehydrogenase (LDH) assay;

FIG. 7A is a photograph of a representative Western blot illustrating phosphorylated Akt and total Akt in Neo-A549 and mtALDH cells under normoxic conditions;

FIG. 7B is a graph illustrating the quantified levels of phosphorylated Akt in Neo-A549 and mtALDH cells under normoxic conditions;

FIG. 7C is a graph illustrating the quantified levels of total Akt in Neo-A549 and mtALDH cells under normoxic conditions;

FIG. 7D is a photograph of a representative Western blot illustrating phosphorylated Akt and total Akt in Neo-A549 and mtALDH cells under prolonged hyperoxic exposure;

FIG. 7E is a graph illustrating the quantified levels of phosphorylated Akt in Neo-A549 and mtALDH cells under prolonged hyperoxic conditions;

FIG. 7F is a graph illustrating the quantified levels of total Akt in Neo-A549 and mtALDH cells under prolonged hyperoxic conditions;

FIG. 8A is a graph illustrating necrotic cell death as measured by a trypan blue exclusion assay in cells pretreated or non-pretreated with LY294002 after 48 hours of normoxic or hyperoxic exposure; and

FIG. 8B is a graph illustrating necrotic cell death as measured by a LDH assay in cells pretreated or non-pretreated with LY294002 after 48 hours of normoxic or hyperoxic exposure.

### DETAILED DESCRIPTION OF THE PREFERRED EMBODIMENT

The following example sets for the preferred embodiments of the present invention. It is to be understood that this example is provided by way of illustration and nothing therein should be taken as a limitation upon the overall scope of the invention.

### EXAMPLE 1

Materials and Methods

Oxygen Exposures: The use of animals in this study was approved by the Institutional Animal Care and Use committee, University of Missouri-Kansas City. The newborn rats at 4 days of age were randomly divided into two groups, room air (normoxia) and oxygen (hyperoxia) exposure groups according to our previous published procedure (34). The animals were housed in regular rat cages that were placed into Lucite chambers. The newborn rats in the chambers breathed either room air or humidified 95% oxygen. Oxygen concentration was monitored continuously with an oxygen analyzer. Dams were given food and water ad libitum, kept on a 12:12 hour on-off light cycle and fostered by rotating in and out of the chamber every 24 hours to avoid oxygen toxicity. At the designated exposure time points, the animals from both treatment groups were sacrificed by exsanguination after receiving intraperitoneal pentobarbital for anesthesia. Lung tissue from each group were collected, minced and stored in liquid nitrogen for protein extraction.

Two Dimensional Gel Electrophoresis and Protein Identification: Protein was extracted from the neonatal rat lungs treated with room air or 95% oxygen. Equal amounts (200 μg) of proteins were resuspended in 200 μL of rehydration buffer containing 8M urea, 2% CHAPS, 0.5% IPG buffer and 0.002% bromophenol blue for isoelectric focusing electrophoresis (IEF). IEF was carried out the IPGphor system from Amersham Bioscience (Piscataway, N.J.). Immobline gel strips (11 cm, pH 3-7, Amersham Bioscience, Piscataway, N.J.) were rehydrated with resuspended samples in rehydration buffer at 30 V, 20° C. for 12 hours (rehydration loading). The gels were run according to the following protocol: 200V, 1 hour; 500V, 1 hour; 1000V, 1 hour; 3000v, 1 hour; gradient from 3000V to 8000V for 3 hours and 8000V, 3 hours. After IEF, Immobline gel strips were equilibrated in buffer containing 50 mM Tris-HCl (pH 6.8), 30% glycerol, 6 M urea, 2% SDS and 1% DTT for 15 minutes at room temperature before being loaded onto sodium dodecyl sulfate-polyacrylamide gel (SDS-PAGE; 8-16%) and sealed with 0.5% agarose gel in 1× Tris/glycine/SDS running buffer with 0.002% bromophenol blue. The electrophoresis was run at 50 mA per gel for approximately two hours. Gels were stained with Bio-Safe Coomassie Satin kit from Bio-Rad Laboratory (Hercules, Calif.) according to manufacturer's protocol. Protein spots on the gels were excised manually in ultra-clean conditions to minimize contamination during gel handling. The gel pieces were destained and residual SDS removed using a solution of acetonitrile and 25 mM ammonium bicarbonate. The gel pieces were then dehydrated with acetonitrile and dried in a vacuum centrifuge. They were hydrated with sequencing-grade modified trypsin and incubated overnight at 37° C. The resulting peptides were extracted out of the gel pieces using a solution of 50% acetonitrile and 5% TFA. Matrix-assisted laser desorption ionization time-of-flight (MALDI-TOF) analysis was performed on an Applied Biosystems Voyager DE-STR mass spectrometer. Samples were spotted onto MALDI plates using an Applied Biosystems SymBiot Sample Workstation. Protein database searching was performed using the accurate molecular weight data provided in the peptide mass map. Peptide masses obtained by MALDI-TOF were entered into the Swiss-Prot and NCBInr protein databases. The Protein Prospector program was used to search for protein candidates.

Plasmid Construction and Transfection: For human mt ALDH plasmid construction, full-length human mtALDH cDNA without stop codon was amplified from a human lung cDNA library (Clontech, Mountain View Calif.) by RT-PCR using following primers, sense: ATGTTGCGCGCTGCCGCCCGCTTC (SEQ ID NO. 1), antisense: TGAGTTCTTCTGAGGCACGAC (SEQ ID NO. 2). The resulting human mtALDH cDNA was subcloned into the plasmid vector pcDNA3.1 (Invitrogen, Carlsbad, Calif.). The mtALDH sequence (SEQ ID NO. 3) was confirmed by direct nucleotide sequencing. mtALDH-pcDNA3 and empty pcDNA3.1 plasmids were transfected into A549 cells using LipofectAMINE (Invitrogen, Carlsbad, Calif.). The transfected cells were then selected by G418 sulfate at 500 μg/mL for ten days. A single clone was selected by limited dilution and mtALDH protein expression was confirmed by Western blotting with anti-V5 antibody (Invitrogen, Carlsbad, Calif.).

Preferably, sequences having the same enzymatic function as mtALDH are also covered by this application. Preferably, such sequences will have at least 80%, more preferably 85%, still more preferably 90%, even more preferably 95%, still more preferably 97%, even more preferably 98%, even more preferably 99%, and most preferably 100% sequence homology or sequence identity with SEQ ID NO. 3. “Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds. Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between gives sequences. Examples of such programs include, but are not limited to, the GCG program package (Devereux, J. et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S., et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95% “sequence identify” to a reference nucleotide sequence, it is intended that the nucleotide sequence of the given polynucleotide is identical to the reference sequence except that the given polynucleotide sequence may include up to 5 point mutations per each 100 nucleotides of the reference nucleotide sequence. In other words, in a polynucleotide having a nucleotide sequence having at least 95% identity relative to the reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95% sequence identity to a reference amino acid sequence, it is intended that the given amino acid sequence of the polypeptide is identical to the reference sequence except that the given polypeptide sequence may include up to 5 amino acid alterations per each 100 amino acids of the reference amino acid sequence. In other words, to obtain a given polypeptide sequence having at least 95% sequence identity with a reference amino acid sequence, up to 5% of the amino acid residues in the reference sequence may be deleted or substituted with another amino acid, or a number of amino acids up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

Similarly, “sequence homology”, as used herein, also refers to a method of determining the relatedness of two sequences. To determine sequence homology, two or more sequences are optimally aligned as described above, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 95% of the amino acid residues or nucleotides in the reference sequence must match or comprise a conservative substitution with another amino acid or nucleotide, or a number of amino acids or nucleotides up to 55 of the total amino acid residues or nucleotides, not including conservative substitutions, in the reference sequence may be inserted into the reference sequence.

A “conservative substitution” refers to the substitution of an amino acid residue or nucleotide with another amino acid residue or nucleotide having similar characteristics or properties including size, hydrophobicity, etc., such that the overall functionality does not change significantly.

Cell culture and Cell Treatment: A549 cells were purchased from American Type culture collection (ATCC, Manassas, Va.) and grown in Dulbecco's Modified Eagle Medium (DMEM) containing 10% fetal bovine serum, 50 μg/mL penicillin and 50 μg/mL streptomycin in 5% CO2 at 37° C. Normoxic exposure of the cells was conducted under room air and 5% CO2 in a humidified cell culture incubator at 37° C. Hyperoxic exposure of the cells was conducted in a humidified chamber (Billups and Rothenberg, Del Mar, Calif.) and the chamber was flushed with 95% O2, 5% CO2 (hyperoxia) at a flow rate of 10 liters per minute for 15 minutes prior to incubation at 37° c. In U0126 and LY294002 pretreatment experiments, cells were treated with or without 10 μM U0216 or LY294002 (two well-known inhibitors) for 30 minutes prior to normoxic or hyperoxic exposure.

Immunofluorescent staining: Cells were cultured on coverslips and fixed with 1% fresh paraformaldehyde in phosphate-buffered saline (PBS) for 15 min. The fixed cells were washed with PBS and permeabilized in 0.2% Triton X-100 in PBS for 5 min. The permeabilized cells were blocked with 1% BSA in PBS for 30 min and stained with anti-V5-FITC antibody (Invitrogen, Carlsbad, Calif.) for one hour. After staining, the coverslips were washed, mounted in mounting medium and viewed under florescent microscope.

Western Blotting Analysis: Antibodies were purchased from Cell Signaling Technology (Beverly, Mass.) and they were used according to manufacturer's instructions. Cultured cells after treatment were washed with cold PBS three times, and the 300 μl of sample lysis buffer (62.5 mM Tris-HCl pH 6.8, 2% w/v SDS, 10% glycerol, 200 mM dithiothreitol, and protease cocktails) was added to each plate. Cell lysates were centrifuged at 12,000×g for 10 minutes. The supernatants were saved for analysis. Protein concentration was determined by bicinchoninic acid (BCA) protein assay kit (Sigma, St. Louis, Mo.). Samples containing 50 μg of protein in loading sample buffer were boiled for 5 minutes and loaded on 12% Tris-Glycine SDS-PAGE gels. Gels were run at 120 V for approximately two hours and transferred overnight at 20 V to nitrocellulose membranes. Membranes were incubated with the blocking buffer containing 5% non-fat mile in PBST (0.1% Tween-20 in PBS) for one hour, washed with PBST and incubated overnight with the primary antibody against either phosphorylated ERK or phosphorylated Akt (Ser473). The membranes were washed in PBST and proteins were visualized using horseradish peroxidase (HRP)-conjugated anti-rabbit IgG and the enhanced chemiluminescence (Amersham Bioscience, Piscataway, N.J.). The membranes were stripped using a standard stripping solution (62.5 mM Tris-HCl, pH 6.8, 2% SDS and 100 mM β-mercaptoethanol) at 50° C., and reprobed with nonphosphorylated ERK, nonphosphorylated Akt and β-actin antibodies. Phosphorylated ERK and phosphorylated Akt protein band intensities on autoradiogram were analyzed with Image-Quant (Molecular Dynamics, Sunnyvale, Calif.) and normalized by nonphosphorylated ERK, nonphosphorylated Akt or β-actin in the same sample, respectively.

mtALDH Activity Assay: mtALDH activity was measured as described previously (9). Neo-A549 and mtALDH A549 cells cultured on plates were collected in buffer of 50 mM Tris-HCl, pH 8.5. Resuspended cells were sonicated at setting 4 for 5 seconds by VirSonic sonicator from VirTis (Gardiner, N.Y.). The cell homogenates were centrifuged at 12,000×g for 10 minutes. The supernatants were saved and protein concentration was determined. Mitochondria were isolated from cultured cells using a mitochondria isolation kit from Pierce (Rockford, Ill.). The enzyme activity assay was carried out in 100 μL of 50 mM Tris-HCl, pH 8.5 containing 50 μg prepared protein, 15 μM propionaldehyde, 1 mM NAD and 1 mM 4-methylpyrazole. The ALDH activity was determined by spectrometer for NADH formation at 340 nm.

Analysis of Necrotic Cell Death (cell viability measurement and cytotoxicity assay): After exposure to normoxic or hyperoxic conditions, non-adherent and trypsinized adherent cells were collected by centrifugation. Both non-adherent and adherent cells were subsequently subjected to staining with trypan blue exclusion (0.2%) for viability within 5 minutes. Cell suspension from each sample was prepared using a 0.4% trypan blue solution in 1:1 dilution. Cells were then loaded onto the counting chambers of a hemocytometer. The number of stained cells and total number of cells were counted at least twice. The cell death was determined by the percentage of stained cells to total cells. The lactate dehydrogenase (LDH) assay kit was from Biovision (Mountain View, Calif.) and LDH activity was measured per manufacture's instruction. Briefly, cells were incubated in an incubator (5% CO2, 37° C.) for the appropriate time of treatment. The cultured media were collected and saved. Adherent cells were washed with PBS and lysed with 1% Triton in 50 mM Tris-HCl, pH7.5. Both cell-cultured media and cell lysates (100 μl/well) were carefully transferred into the corresponding wells of a 96-well plate. Reaction Mixture (100 μl) was then added to each well and incubated for 30 minutes at room temperature. The absorbance of all samples at 490 nm was measured using a microplate reader. The cytotoxicity was determined by the percentage of LDH activity in cultured medium over combined LDH activities of the cultured medium and cell lysate.

Analysis of Apoptotic Cells: The Apoptosis Detection kit was from R&D System (Minneapolis, Minn.). Treated cells were trypsinized and collected by centrifugation at 500×g for 5 minutes. Cells were washed with cold PBS once and resuspended in 100 uL binding buffer containing 10 mM HEPES pH 7.4, 150 mM NaCl, 5 mM KCl, 1 mM MgCl2 and 1.8 mM CaCl2. Cells were stained with Annexin V-FITC (0.05 μg per sample) for 15 minutes according to manufacturer's instructions. The stained cells were then subjected to flow cytometry analysis.

Assessment of Intracellular and Mitochondrial ROS Levels: After normoxic or hyperoxic treatment, cultured cells were stained with 10 μM of 2′,7′-dicholordihydrofluorescein diacetate, succinimidyl ester (OxyBURST® Green H2DCFDA (2′,7′-dichlorodihydrofluorescin diacetate)), SE, Molecular Probe, Oreg.) for 10 minutes. The stained cells were washed three times with PBS and then trypsinized with 0.025% trypsin and 0.05% EDTA. The resuspended cells were subjected to fluorescent intensity measured by flow cytometry. For mitochondria-derived ROS measurement, cells were incubated with 10 μM dihydrorhodamaine 123 (Molecular Probe, OR) for 15 minutes. The stained cells were washed three times with PBS and then trypsinized with 0.25% trypsin, 0.05% EDTA. The resuspended cells were subjected to rhodamine 123 fluorescent intensity measurement by flow cytometry. Statistical Analysis: The results are expressed as the mean±SEM of data obtained from two or more experiments; or where appropriate, as mean±SD. Statistical analysis was performed using the student t test for paired comparisons and ANOVA for multiple comparisons. A value of p<0.05 was considered significant.

### RESULTS

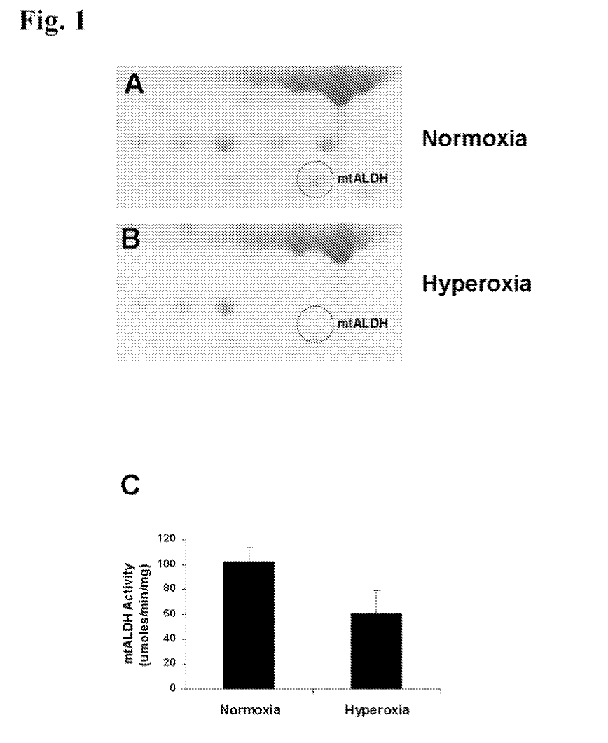

The protein extracts from neonatal rat lung tissue after 10 days of normoxic or hyperoxic (95% O₂) exposure were analyzed by two dimensional gel electrophoresis (2-DE). Many protein spots were displayed on the gels from pI 3 to 7 (data not shown). Six unknown protein spots, one gel blank spot, and one positive control spot (serum albumin) were excised from the Coomassie blue stained gels for protein identification. One of the unknown and down-regulated protein spots (FIGS. 1A and 1B, circled) was identified as a nuclear-encoded mtALDH. mtALDH appeared as a discrete spot (pI=6.0, MW=56.0) on the gels of the normoxic group and the same protein was not visible on the gels of hyperoxic group (FIG. 1B). mtALDH activities were measured in isolated mitochondria from cultured A549 lung type II epithelial cells treated with normoxia or hyperoxia for 3 days. The mtALDH activity in hyperoxia-treated A549 cells was decreased by approximately 40% compared to normoxia-treated A549 cells (n=3; FIG. 1C). Isolated mitochondrial protein from attached cells was used for the mtALDH activity assay.

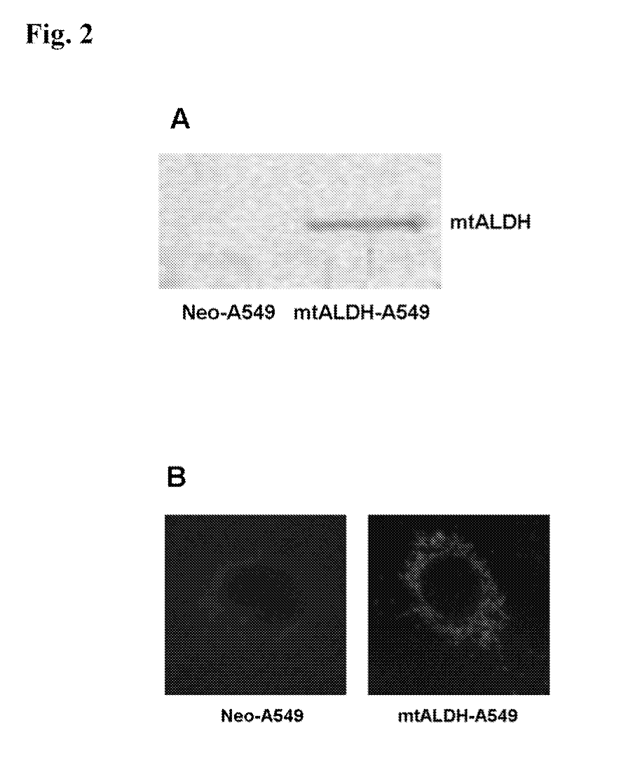

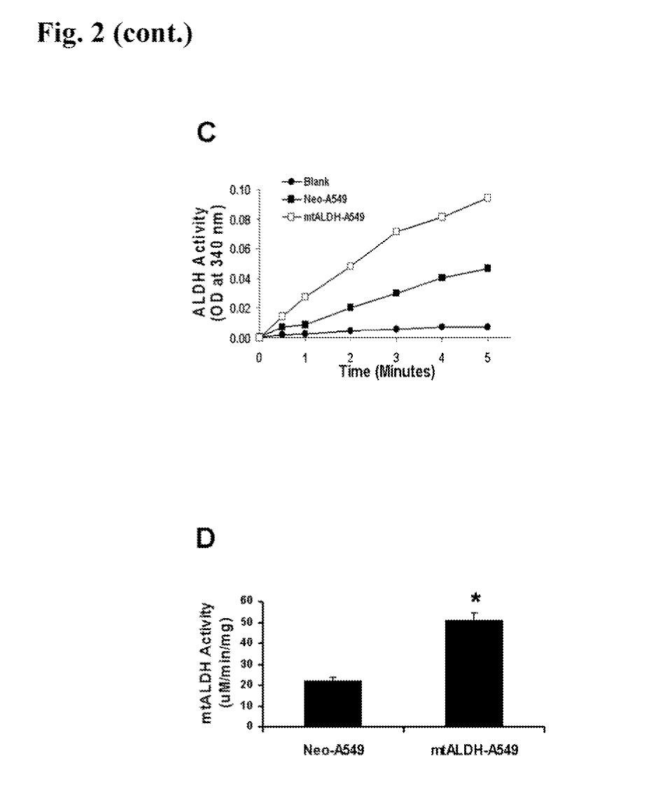

To characterize the role of mtALDH in hyperoxic lung injury and cell death, we generated a stable cell line (mtALDH-A549) overexpressing human mtALDH-V5 fusion protein by transfecting pcDNA3-V5-human mtALDH plasmid into A549 lung type II epithelial cells. mtALDH overexpression was detected with an anti-V5 antibody by Western blotting in mtALDH-A549 cells, but not in control Neo-A549, as shown in FIG. 2A. An immunofluorescent study of mtALDH-A549 cells with an anti-V5 antibody revealed a punctuate appearance in cytoplasm, which is consistent with mitochondrial distribution. No specific immunofluorescent staining was observed in the cytoplasm of Neo-A549 cells (FIG. 2B). Total mtALDH activities were also assayed in Neo-A549 and mtALDH-A549 cells (FIG. 2C). The total activity increased more than two fold in mtALDH-A549 cells compared to Neo-A549 cells (p<0.01, n=6; FIG. 2D).

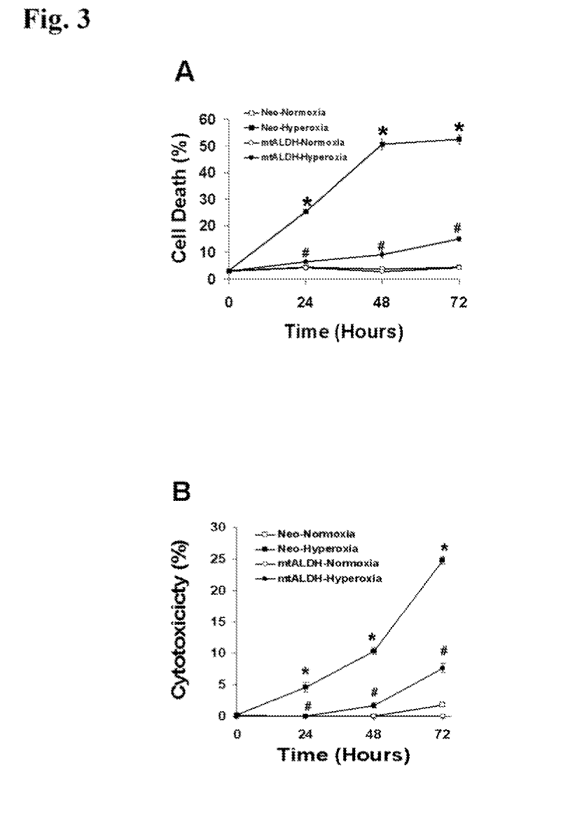

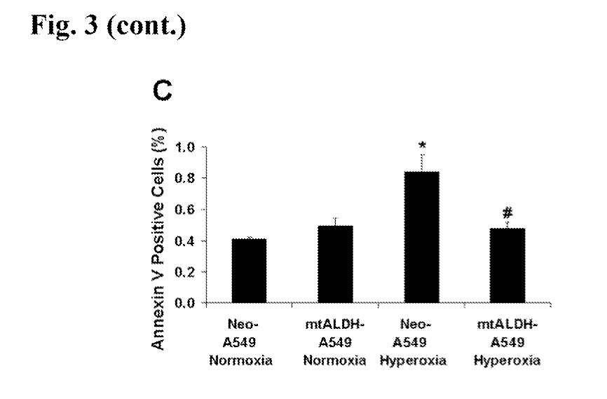

Apoptotic and necrotic cell death during hyperoxia is though to be responsible for acute and chronic lung injury. In this experiment, Neo-A549 and mtALDH-A549 cells were cultured under conditions of normoxia and hyperoxia and necrotic cell death was measured by trypan blue exclusion and cytotoxicity assays. After normoxic exposure for up to 72 hours, necrotic cell death between Neo-A549 and mtALDH-A549 cells was similar and ranged from 2.8% to 4.5% in a trypan blue exclusion assay (FIG 3A) and from 0% to 1.7% in an LDH cytotoxicity assay (FIG. 3B). Hyperoxia caused significantly increased necrotic cell death in Neo-A549 cells. The dead cells could be found in both non-adherent and adherent cells in trypan blue exclusion assay. Compared to Neo-A549 cells under normoxic conditions, the percentage of necrotic cell death under hyperoxic conditions increased from 4.5% to 25.3% after 24 hours, from 3.7% to 50.5% after 48 hours, and from 4.5% to 52.4% after 72 hours (p<0.001, n=6; FIG. 3A). In a cytotoxicity assay, the percentage of cytotoxicity in Neo-A549 cells increased to 4.6% from 0%, to 10.3% from 0% and 24.8% from 1.7% after 24, 48 and 72-hour hyperoxic exposure, respectively, compared to the cells exposed to normoxia (p<0.001, n=6; FIG. 3B). The cytotoxicity was presented by the percentage of LDH activity in cultured medium compared with combined LDH activities from both cultured medium and cell lysate. The apoptotic cell death after 48-hour normoxic or hyperoxic exposure was analyzed by Annexin V staining and flow cytometry (FIG. 3C). The percentage of Annexin V positive cells was significantly higher in hyperoxia-treated Neo-A549 cells (0.84%) than in normoxia-treated Neo-A549 cells (0.41%; p<0.01, n=6).

When mtALDH-A549 cells were treated with the same hyperoxic conditions, the percentage of hyperoxia-induced necrotic cell death in mtALDH-A549 cells was significantly lowered compared to Neo-A549 cells in trypan blue exclusion assay (FIG. 3A). The necrotic cell death decreased to 6.5% in mtALDH-A549 cells from 25.3% in Neo-A549 cells (p<0.001, n=6), to 9.1% from 50.5% (p<0.001, n=6) and to 15.1% from 52.4% (p<0.001, n=6) after 24, 48 and 72-hour hyperoxic exposure, respectively. The percentage of necrotic cell death in cytotoxicity assay after hyperoxic exposure in mtALDH-A549 was also significantly decreased when compared to Neo-A549 cells (FIG. 3B). The necrotic cell death was decreased to 0% in mtALDH-A549 cells from 4.7% in Neo-A549 cells after 24 hours (p<0.001, n=6), to 1.7% from 10.3% after 48 hours (p<0.001, n=6) and to 7.6% from 24.8% after 72 hours (p<0.001, n=6). The percentage of apoptotic cell death assayed by Annexin V staining was significantly lowered to 0.48% in mtALDH-A549 cells from 0.84% in Neo-A549 cells after 48-hour hyperoxic treatment (p<0.001, n=6; FIG. 3C). Alterations of DNA fragmentation, cytochome c release, or caspase 3 and 9 activation were not observed after normoxic or hyperoxic treatment in cultured Neo-A549 or mtALDH-A549 cells (data not shown).

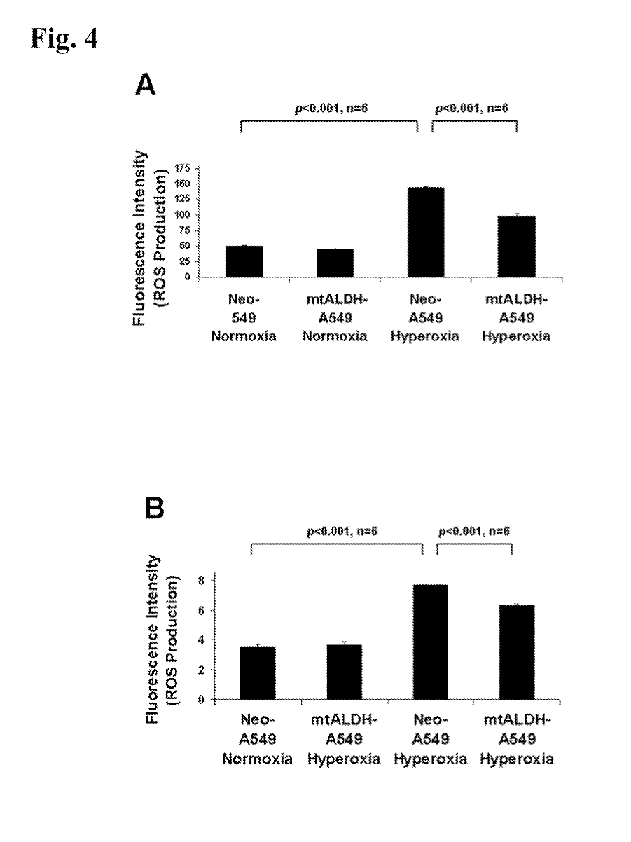

Intracellular ROS levels were measured by flow cytometry after the cultured cells were stained with H2DCFDA (FIG. 4A). The intracellular ROS levels were similar in Neo-A549 and mtALDH-A549 cells under normoxic conditions (room air and 5% CO₂). After 24-hour hyperoxic exposure (95% O₂ and 5% CO₂), the intracellular ROS level in Neo-A549 cells increased approximately three-fold compared to the cells exposed to normoxia (p<0.001, n=6). However, the intracellular ROS level in mtALDH-A549 increased only approximately two fold compared to Neo-A549 cells after 24-hour hyperoxia treatment. The intracellular ROS level in mtALDH-A549 cells was significantly decreased compared to Neo-A549 cells (p<0.001, n=6). Mitochondria-derived ROS levels were measured by flow cytometry after the cells were stained with dihydrorhodamine 123 (FIG. 4B). The mitochondrial ROS levels in Neo-A549 and mtALDH-A549 cells were similar under normoxic conditions. The mitochondrial ROS level in Neo-A549 cells after 24-hour hyperoxic exposure increased approximately two fold compared to the cells exposed to normoxia (p<0.001, n=6). The mitochonfrial ROS level in mtALDH-A549 cells was also increased compared to cells under hyperoxic conditions, but its level was significantly decreased compared to Neo-A549 cells (p<0.001, n=6).

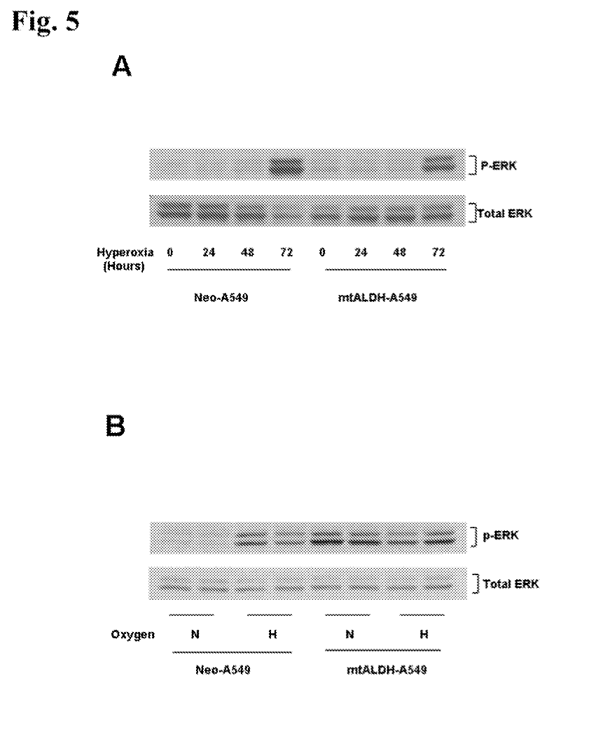

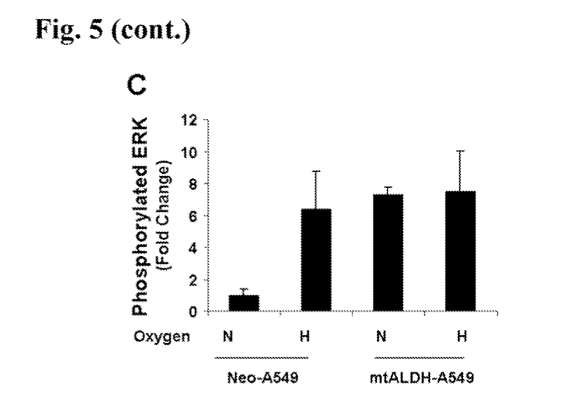

Western blotting analysis showed that both mtALDH and hyperoxia stimulated ERK phosphorylation. ERK activation was detected in mtALDH-A549 cells after 0, 24, 48 and 72-hour hyperoxic exposure (95% O₂ and 5% CO₂) and in Neo-A549 cells after 48 and 72-hour hyperoxic exposure (95% O₂ and 5% CO₂) (FIG. 5A). Under 48-hour normoxic conditions (room air and 5% CO₂), phosphorylated ERK in Neo-A549 cells was expressed at a very low level. However, mtALDH stimulated ERK phosphorylation in mtALDH-A549 cells under the same normoxic conditions. A seven-fold increase in ERK phosphorylation in mtALDH-A549 cells was detected compared to Neo-A549 cells (FIGS. 5B and 5C). Hyperoxia also stimulated a six-fold increase in ERK phosphorylation in Neo-A549 cells after a 48-hour hyperoxic exposure. The ERK phosphorylation after a 48-hour hyperoxic exposure in mtALDH-A549 cells was maintained at a high level that was similar to the level prior to hyperoxic exposure (FIGS. 5B and 5C wherein the levels of phosphorylated ERK in FIG. 5B were quantified by densitometry and normalized by total ERK with the data being expressed as mean±SD).

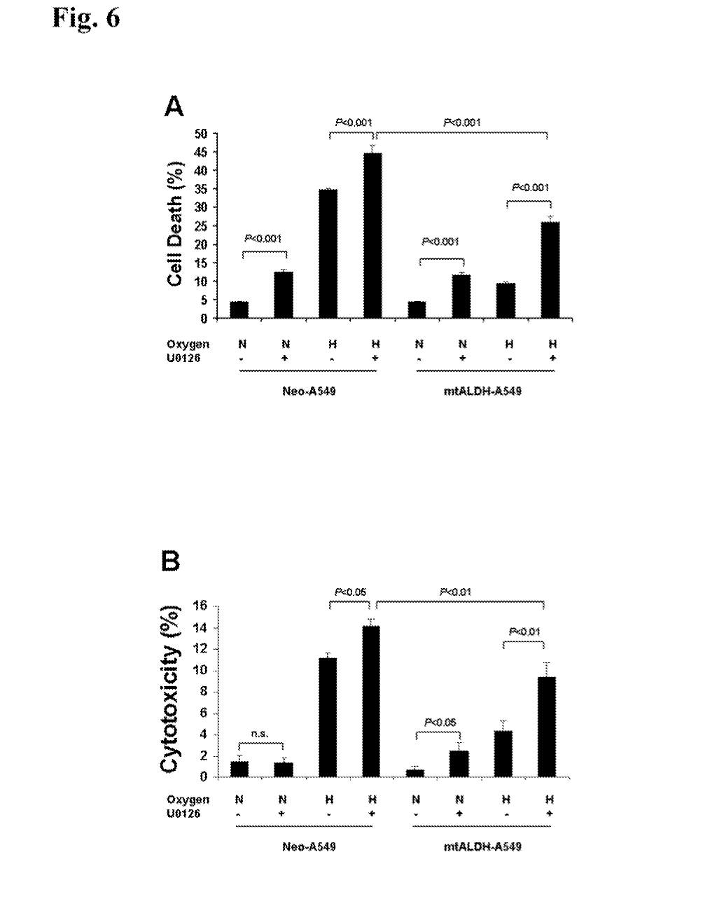

Next, pretreated Neo-A549 and mtALDH-A549 cells with or without 10 μM U0126, an upstream kinase (MEK1/2) inhibitor, were measured for necrotic cell death by trypan blue exclusion and cytotoxicity assays after 48-hour normoxic or hyperoxic exposure. The U0126 pretreatment increased the necrotic cell death in Neo-A549 and mtALDH-549 cells after 48-hour normoxic (room air and 5% CO₂) or hyperoxic (95% O₂ and 5% CO₂) treatments. The necrotic cell death measured by trypan blue exclusion assay in Neo-A549 cells after U0126 pretreatment significantly increased to 12.6% from 4.6% under normoxic conditions (p<0.001, n=6; FIG. 6A), and to 44.5% from 34.7% under hyperoxic conditions (p<0.001, n=6; FIG. 6A). In an LDH cytotoxicity assay, the necrotic cell death in Neo-A549 cells after U0126 pretreatment increased to 14.1% from 11.2% under hyperoxic conditions (p<0.05, n=6; FIG. 6B). The necrotic cell death measured by trypan blue exclusion assay in mtALDH A549 cells after U0126 pretreatment increased to 11.6% from 4.7% under normoxic conditions (p<0.001, n=6; FIG 6A), to 26.0% from 9.3% under hyperoxic conditions (p<0.001, n=6; FIG. 6A). In an LDH cytotoxicity assay, the necrotic cell death in mtALDH-A549 cells after U0126 pretreatment increased to 9.4% from 4.3% under hyperoxic conditions (p<0.01, n=6; FIG. 6B). The necrotic cell death after hyperoxic exposure in U0126 pretreated mtALDH-A549 cells was significantly lower than that in U0126-pretreated Neo-A549 cells (p<0.001, n=6; FIGS. 6A and 6B).

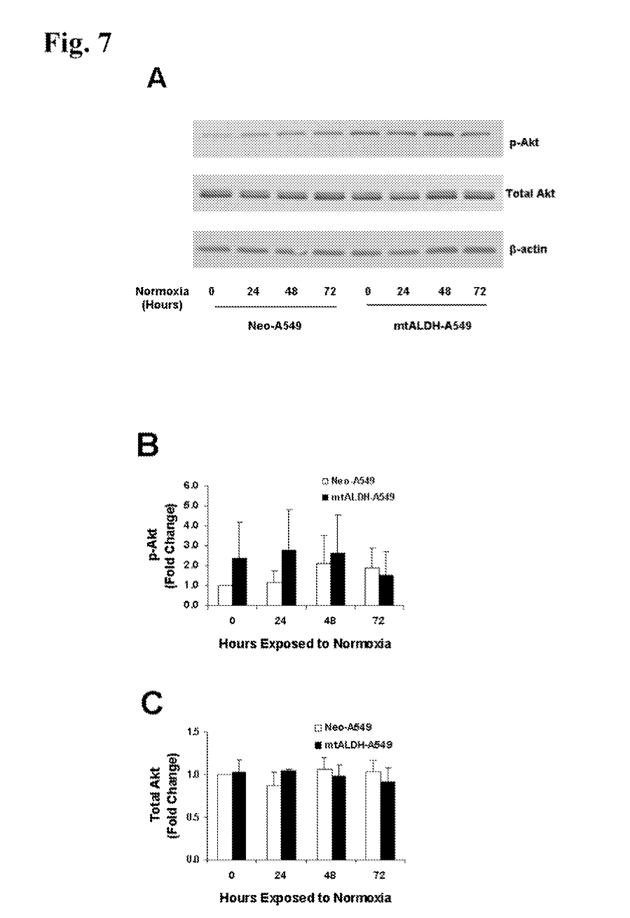

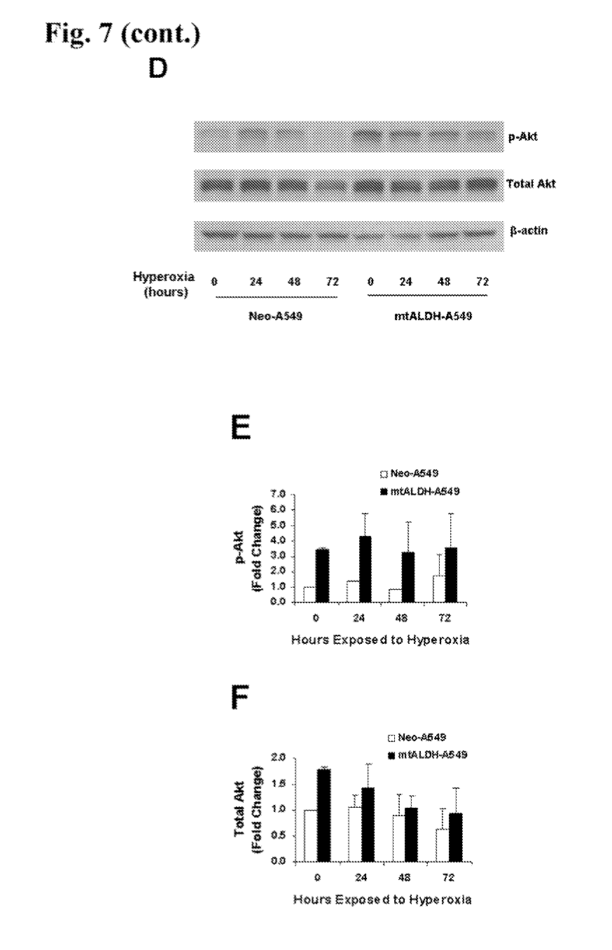

PI3K/Akt activation was analyzed in Neo-A549 and mtALDH-A549 cells by Western blotting. Under normoxic conditions (room air and 5% CO₂), mtALDH stimulated Akt phosphorylation. The phosphorylated Akt level was two-fold higher in mtALDH-A549 cells than that in Neo-A549 cells during the first 24-hour culture under normoxic conditions (FIGS. 7A and 7B). The total Akt levels in Neo-A549 and mtALDH-A549 cells were not significantly changed under normoxic conditions (FIGS. 7A and 7C). For Figs. B and C, and E and F, levels of phosphoryated Akt and total Akt from two separated experiments under both normoxic and hyperoxic conditions were quantified by densitometry and normalized by β-actin. Data were expressed as mean±SD. Under hyperoxic conditions (95% O₂ and 5% CO₂), phosphorylated Akt was slightly increased (FIG. 7D) and total Akt level was not significantly altered (FIG. 7E) in Neo-A549 cells. However, Akt phosphorylation was approximately 2-3 times higher in mtALDH-A549 cells than that in Neo-A549 cells during 0, 24 and 48-hour hyperoxic exposure (FIG. 7D). Prior to hyperoxic treatment (0 hour), total Akt was increased about 1.8 fold in mtALDH-A549 cells compared to Neo-A549 cells. The total Akt was not significantly altered in Neo-A549 and mtALDH-A549 cells during 24, 48 and 72-hour hyperoxic exposure (FIGS. 7E and 7F).

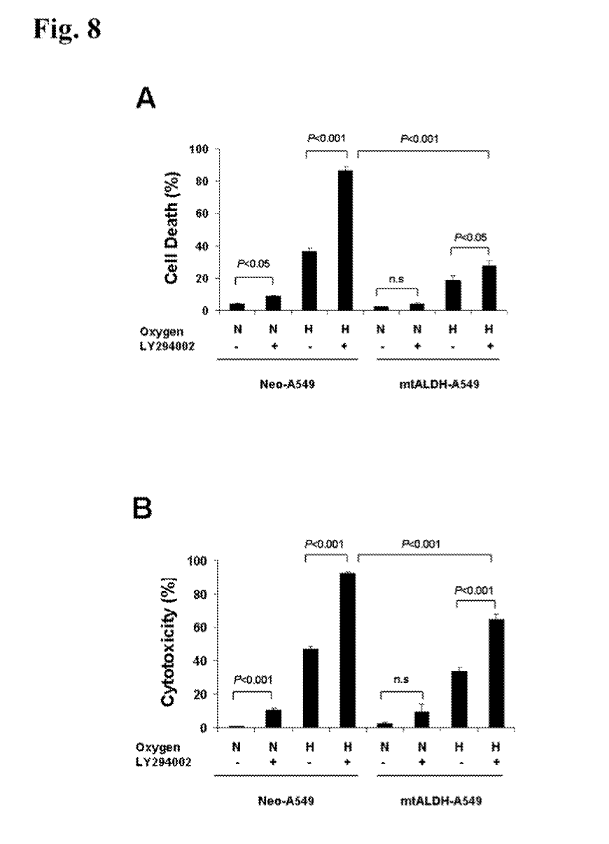

Next, Neo-A549 and mtALDH-A549 cells were pretreated with or without 10 μM LY294002, a PI3K inhibitor, to inactivate PI3K. Necrotic cell death was measured by trypan blue exclusion and cytotoxicity assays after 48-hour normoxic (room air and 5% CO₂) or hyperoxic exposure (95% O₂ and 5% CO₂). The LY294002 pretreatment increased the necrotic cell death in Neo-A549 and mtALDH-A549 cells after 48-hour normoxic or hyperoxic treatment. The necrotic cell death measured by trypan blue exclusion assay in LY294002 pretreated Neo-A549 cells significantly increased to 9.0% from 4.2% under normoxic conditions (p<0.05, n=6; FIG. 8A), and to 86.6% from 36.7% under hyperoxic conditions (p<0.001, n=6; FIG. 8A). In an LDH cytotoxicity assay, the necrotic cell death in LY294002 pretreated Neo-A549 cells increased to 10.4% from 0.7% under normoxic conditions (p<0.001, n=6; FIG. 8B), to 92.4% from 46.9% under hyperoxic conditions (p<0.001, n=6; FIG. 8B). The necrotic cell death measured by trypan blue exclusion assay in LY294002 pretreated mtALDH-A549 cells increased to 4.3% from 2.3% under normoxic conditions (n.s, n=6; FIG. 8A), to 28.0% from 18.7% under hyperoxic conditions (p<0.05, n=6; FIG. 8A). In an LDH cytotoxicity assay, the necrotic cell death in LY294002 pretreated mtALDH-A549 cells increased to 8.9% from 2.5% under normoxic conditions (room air and 5% CO₂) (n.s, n=6; FIG. 8B), to 64.4% from 33.9% under hyperoxic conditions (95% O₂ and 5% CO₂) (p<0.001, n=6; FIG. 8B). The necrotic cell death in LY294002 pretreated mtALDH-A549 cells after hyperoxic exposure was significantly lower than that in LY294002 pretreated Neo-A549 (p<0.001, n=6; FIGS. 8A and 8B).

### DISCUSSION

The present study demonstrated that hyperoxia down-regulated mtALDH in the neonatal rat lung. In cultured lung epithelial cells, hyperoxia induced both apoptotic and nonapoptotic cell death. mtALDH overexpression in lung epithelial cells conferred cellular resistance to hyperoxia and significantly attenuated hyperoxia-induced cell death. The ROS production in cultured lung epithelial cells was elevated after hyperoxic exposure. Overexpression of mtALDH decreased intracellular and mitochondria-derived ROS production, indicating that mtALDH might have antioxidant and cytoprotective effects. mtALDH overexpression significantly stimulated ERK/MAPK and PI3/Akt activation under normoxic or hyperoxic conditions. Inhibition of ERK/MAPK and PI3K/Akt activation eliminated cytoprotective effects of mtALDH, suggesting that mtALDH might activate ERK/MAPK and PI3K/Akt signaling pathways which in turn exerts a cytoprotective role in cell survival during hyperoxia.

mtALDH is a nuclear encoding mitochondrial protein, localized in mitochondrial matrix. mtALDH is a reductase of acetaldehyde and converts acetaldehyde to acetic acid (25). It has been reported previously that deficiency of mtALDH increases cell susceptibility to oxidative stress and it also increases the risks in the development of Alzheimer's disease (23, 24). Overexpression of mtALDH may detoxify acetaldehyde and prevent acetaldehyde-induced cell injury in human umbilical vein endothelial cells (19). mtALDH is expressed in the lung (44), but its role in lung injury is not clear. Proteomic analysis in this study revealed that mtALDH was down-regulated in the neonatal rat lungs after hyperoxic exposure. This finding indicates that mtALDH may be implicated in oxidative stress and cell death in hyperoxic lung injury.

Lung injury due to supplemental oxygen therapy is characterized by the extensive pulmonary cell death (3, 5, 8). Hyperoxia induces lung epithelial cell death by activating apoptotic and nonapoptotic cell death pathways. Apoptosis in lung epithelial cells induced by hyperoxia is a highly regulated process. Hyperoxia can trigger either death receptor or mitochondria-mediated apoptosis pathway. For instance, hyperoxia induces apoptosis in lung epithelial cells via activation of Fas/FasL (12), increases cytochrome c release from mitochondria (27), or activation of caspases (6). In the cultured human lung type II epithelial cell line (A549), hyperoxia primarily induces necrotic cell death, though a small percentage of cell death may be due to apoptosis (13, 18, 21, 40). The results herein also revealed that hyperoxia induced both apoptotic and nonapoptotic cell death in A549 lung epithelial cells, which is consistent with previous findings by other groups (13, 18, 21, 40). The prevention of cell death against hyperoxia in lung epithelial cells has been investigated extensively for its potentially therapeutic use. Previous reports have demonstrated that growth factors (granulocyte macrophage-colony stimulating factor and keratinocyte growth factor (28, 30), and antioxidant enzymes (heme oxygenase-1 and superoxide dismutase) (2, 33, 41, 42), have therapeutic effects on oxidative stress related conditions, including hyperoxic lung injury. One of the important findings in this study was that overexpression of human mtALDH in A549 cells significantly reduced hyperoxia-induced apoptotic and nonapoptotic cell death. Thus, it may be valuable to maintain an adequate level of mtALDH to aid in the prevention and treatment of hyperoxic lung injury.

Hyperoxia increase ROS production in lung epithelial cells. The increased ROS level is primarily generated from mitochondria and other oxidases such as NADH oxidase (4, 38, 43). An increase in ROS is extremely toxic and causes cell death and lung injury (8). Reduced ROS by antioxidants after hyperoxic exposure decreases cell death and lung injury (3). Our data demonstrated that mtALDH overexpression could reduce both intracellular and mitochondria-derived ROS production in lung epithelial cells during hyperoxic exposure. The reduced ROS in mtALDH-A549 cells may delay hyperoxia-induced cell death.

The activation of the ERK/MAPK pathway has been previously reported in lung epithelial cells after hyperoxic exposure. ERK activation in lung epithelial cells has a protective effect in hyperoxia-induced cell death and it prolongs cell survival (7, 31, 39). For example, overexpression of 8-oxoguanine DNA glycosylase (hOggl), a base excision DNA repair protein, protected against hyperoxia-induced cell death via activation of ERK in A549 lung epithelial cells (17). The activation of ERK signaling after hyperoxic exposure has also been reported to increase Nrf2 translocation and antioxidant response element (ARE)-mediated gene expression involved in cellular protection (29). A recent report has indicated that down-regulated phosphatase increases ERK/MAPK phosphorylation and reduces macrophage cell death after hyperoxic exposure (45). It is not known whether the activation of ERK/MAPK by hyperoxia in lung epithelial cells is due to down-regulation of phosphatase or through other pathways. The data found herein further confirmed that hyperoxia activated ERK/MAPK signaling pathways as a result of cellular response to oxidative stress. Additionally, it was found that overexpression of mtALDH activated ERK/MAPK cell survival signaling under both normoxic and hyperoxic conditions. Activation of ERK/MAPK signaling by mtALDH attenuated hyperoxia-induced cell death and increased cell survival. When the activation of ERK/MAPK was inhibited by the MEK1/2 inhibitor, U0126, there was increased necrotic cell death in Neo-A549 and mtALDH-A549 cells after hyperoxic exposure. However, the cell death after ERK/MAPK inactivation in mtALDH-A549 cells was significantly lower than that in Neo-A549 cells, suggesting that ERK/MAPK activation by mtALDH may have a correlation with the cytoprotective effects and cell survival in lung epithelial cells.

The Akt cell survival pathway is implicated in hyperoxia-induced cell death in lung epithelial cells. It has been reported that prolonged hyperoxia not only diminishes Akt phosphorylation, but also down-regulates total Akt protein, which is one of the possible causes in hyperoxia-induced cell death (39). The data generated herein demonstrates that mtALDH overexpression in A549 lung epithelial cells stimulates Akt activation under normoxic conditions. The activated Akt and total Akt are retained in mtALDH-A549 cells even under hyperoxic conditions. Constitutive expression of the active form of Akt has been shown to increase mouse survival under hyperoxic conditions (1, 20). Overexpression of growth factors, such as keratinocyte growth factor, increases Akt kinase activity and inhibits Fas/FasL-mediated apoptosis in lung epithelial cells (28, 30). Most recently, it has been demonstrated that overexpression of Cyr61, a novel stress-related protein, exerts cytoprotection in hyperoxia-induced pulmonary epithelial cell death; an effect mediated in part via the Akt signaling pathway (16). This study also demonstrated that inhibition of PI3K accelerated cell death in the lung epithelial cells that overexpressed mtALDH, suggesting that PI3K activation is required for the cytoprotective effect of mtALDH in the lung epithelial cells. Since PI3K activation leads to activation of Akt and several other downstream effectors such as PKC zeta, PKC delta, and ERK, more specific Akt inhibitor studies are needed to provide conclusive information about the role of Akt in the cytoprotective mechanisms of mtALDH.

In the present study, it is still unclear how mtALDH overexpression activates ERK and Akt cell survival signaling pathways, however, the activation is measurable. Hyperoxia induces ERK and Akt activation following hyperoxic exposure. The mechanisms of ERK and Akt activation by mtALDH might be different from hyperoxia-induced ERK and Akt activations, since ERK and Akt activation by mtALDH overexpression is prior to hyperoxic exposure without significant ROS alteration under the experimental conditions herein. mtALDH is a key enzyme in ethanol metabolism and is also involved in detoxification of aldehyde. Aldehyde is a toxic substance and a deficiency of mtALDH would cause accumulation of aldehyde in cells, which would induce oxidative stress and result in protein and lipid dysfunction. Further studies are needed to investigate how mtALDH overexpression activates ERK and Akt in lung epithelial cells.

The teachings and content of the following reference are incorporated by reference herein:
1. Ahmad S, Ahmad A, Gerasimovskaya E, Stenmark K R, Allen C B, White C W. Hypoxia protects human lung microvascular endothelial and epithelial-like cells against oxygen toxicity: role of phosphatidylinositol 3-kinase. Am J Respir Cell Mol Biol. 28:179-87, 2003.
2. Ahmed M N, Suliman H B, Folz R J, Nozik-Grayck E, Golson M L, Mason S N, Auten R L. Extracellular superoxide dismutase protects lung development in hyperoxia-exposed newborn mice. Am. J. Respir. Crit. Care Med. 167:400-538-2003.
3. Asikainen T M, White C W. Pulmonary antioxidant defenses in the preterm newborn with respiratory distress and bronchopulmonary dysplasia in evolution: implications for antioxidant therapy. Antioxid Redox Signal. 6:155-67, 2004.
4. Balaban R S, Nemoto S, Finkel T. Mitochondria, oxidants, and aging. Cell 120:483-95, 2005.
5. Bourbon J, Boucherat O, Chailley-Heu B, Delacourt C. Control mechanisms of lung alveolar development and their disorders in bronchopulmonary dysplasia. Pediatr Res. 57:38R-46R, 2005.
6. Buccellato L J, Tso M, Akinci O I, Chandel N S, Budinger G R. Reactive oxygen species are required for hyperoxia-induced Bax activation and cell death in alveolar epithelial cells. J Biol Chem. 279:6753-60, 2004.
7. Buckley, S, Driscoll B, Barsky L, Weinberg K, Anderson K, Warburton D. ERK activation protects against DNS damage and apoptosis in hyperoxic rat AEC2. Am J Physiol. 277:L159-66, 1999.
8. Chabot F, Mitchell J A, Gutteridge J M, Evans T W. Reactive oxygen species in acute lung injury Eur Respir J. 11:745-57, 1998.
9. Chen, A, Zhang J, Stamler J S. Identification of the enzymatic mechanism of nitroglycerin bioactivation. Proc Natl Acad Sci USA, 99: 8306-8311, 2002.
10. Chiu D, Ma K, Scott A, Duronio V. Acute activation of Erk1/Erk2 and protein kinase B/akt proceed by independent pathways in multiple cell types. FEBS J. 272:4372-84, 2005.
11. Comhair S A, Erzurum S C. Antioxidant responses to oxidant-mediated lung diseases. Am J Physiol Lung Cell Mol Physiol. 283:L246-55, 2002.
12. De Paepe M E, Mao Q, Chao Y, Powell J L, Rubin L P, Sharma S. Hyperoxia-induced apoptosis and Fas/FasL expression in lung epithelial cells. Am J Physiol Lung Cell Mol Physiol. 289:L647-59, 2005.
13. Franek W R, Morrow D M, Zhu H, Vancurova I, Miskolei V, Carley-Usmar K, Simms H H, Mantell L L. NF-kappaB protects lung epithelium against hyperoxia-induced nonapoptotic cell death-oncosis. Free Radic Biol Med. 37:1670-9, 2004.
14. Frank L, Groseclose E E. Preparation for birth into an O2-rich environment: the antioxidant enzymes in the developing rabbit lung. Pediatr Res 18:240-4, 1984.
15. Janssen Y M, Van Houten B, Borm P J, Mossman B T. Cell and tissue responses to oxidative damage. Lab Invest. 69:261-74, 1993.
16. Jin Y, Kim H P, Ifedigbo E, Lau L F, Choi A M. Cry61 protects against hyperoxia-induced cell death via Akt pathway in pulmonary epithelial cells. Am J Respir Cell Mol Biol. 33:297-302, 2005.
17. Kannan S, Pang H, Foster D C, Rao Z, Wu M. Human 8-oxoguanine DNA glycosylase increases resistance to hyperoxic cytotoxicity in lung epithelial cells and involvement with altered MAPK activity. Cell Death Differ. 13:311-23, 2005.
18. Kazzaz J A, Xu J, Palaia T A, Mantell L, Fein A M, Horowitz S. Cellular oxygen toxicity. Oxidant injury without apoptosis. J Biol chem. 271:15182-6, 1996.
19. Li S Y, Gomelsky, M, Duan J, Zhang Z, Gomelsky L, Zhang X, Epstein P N, Ren J. Overexpression of aldehyde dehydrogenase-2 (ALDH2) transgene prevents acetaldehyde-induced cell injury in human umbilical vein endothelial cells: role of ERK and p38 mitogen-activated protein kinase.J Biol Chem. 279:11244-52, 2004.
20. Lu Y, Parkyn L, Otterbein L E, Kureishi Y, Walsh K, Ray A, Ray P. Activated Akt protects the lung from oxidant-induced injury and delays death of mice. J Exp Med. 193:545-49, 2001.
21. Mantell L L, Horowitz S, Davis J M, Kazzaz J A. Hyperoxia-induced cell death in the lung—the correlation of apoptosis, necrosis, and inflammation. Am NY Acad Sci. 887:171-80, 1999.
22. Martin T R, Hagimoto N, Nakamura M, Matute-Bello G. Apoptosis and epithelial injury in the lungs. Proc Am Thorac Soc. 2:214-20, 2005.
23. Ohsawa I, Nishimaki K, Yasuda C, Kamino K, Ohta S. Deficiency in a mitochondrial aldehyde dehydrogenase increases vulnerability to oxidative stress in PCl2cells. J Neurochem. 84:1110-7, 2003.
24. Ohta S, Ohsawa I, Kamino K, Ando F, Shimokata H. Mitochondrial ALDH2 deficiency as an oxidative stress. Ann NY Acad Sci. 1011:36-44, 2004.
25. Oyama T, Isse T, Kagawa N, Kinaga T, Kim Y D, Morita M, Sugio K, Weiner H, Yasumoto K, Kawamoto T. Tissue-distribution of aldehyde dehydrogenase 2 and effects of the ALDH2 gene-disruption on the expression of enzymes involved in alcohol metabolism. Front Biosci. 10:951-60, 2005.
26. Pagano A, Barazzone-Argiroffo C. Alveolar cell death in hyperoxia-induced lung injury. Ann NY Acad Sci. 1010:405-16, 2003.
27. Pagano A, Donati Y, Metrailler I, Barazzone Argiroffo C. Mitochondrial cytochrome c release is a key event in hyperoxia-induced lung injury: protection by cyclosporin A. Am J Physiol Lung Cell Mol Physiol. 286:L275-83, 2004.
28. Paine R3rd, Wilcoxen S E, Morris S B, Sartori C, Baleeiro C E, Matthay M A, Christensen P J. Transgenic overexpression of granulocyte macrophage-colony stimulating factor in the lung prevents hyperoxic lung injury. Am J Pathol. 163:2397-406, 2003.
29. Papaiahgari S, Kleeberger S R, Cho H Y, Kalvakolanu D V, Reddy S P. NADPH oxidase and ERK signaling regulates hyperoxia-induced Nrf2-ARE transcriptional response in pulmonary epithelial cells. J Biol Chem. 279:42302-12, 2004.
30. Ray P, Devaux Y, Stolz D B, Yarlagadda M, Watkins S C, Lu Y, Chen L, Yang X F, Ray A. Inducible expression of keratinocyte growth factor (KGF) in mice inhibits lung epithelial cell death induced by hyperoxia. Proc Natl Acad Sci USA. 100:6098-103, 2003.
31. Romashko J 3rd, Horowitz S, Franek W R, Palaia T, Miller E J, Lin A, Birrer M J, Scott W, Mantell L L, MAPK pathways mediate hyperoxia-induced oncotic cell death in lung epithelial cells. Free Radic Biol Med. 35:978-93, 2003.
32. Ruchko M, Gorodnya O, LeDoux S P, Alexeyev M F, Al-Mehdi A B, Gillespie M N. Mitochondrial DNA damage triggers mitochondrial dysfunction and apoptosis in oxidant-challenged lung endothelial cells. Am J Physiol Lung Cell Mol Physiol. 288:L530-5, 2005.
33. Ryter S W, Choi A M. Heme oxygenase-1: redox regulation of a stress protein in lung and cell culture models. Antioxid Redox Signal. 7:80-91, 2005.
34. Shaffer S G, Bradt S K, Thibeault D W. Chronic vascular pulmonary dysplasia associated with neonatal hyperoxia exposure in the rat. Pediatr. Res. 21:14-20, 1987.
35. Sigaud S, Evelson P, Gonzalez-Flecha B. H2O2-induced proliferation of primary alveolar epithelial cells in mediated by MAP kinases. Antioxid Redox Signal. 7:6-13, 2005.
36. Spragg R G. DNA strand break formation following exposure of bovine pulmonary artery and aortic endothelial cells to reactive oxygen products. Am J Respir Cell Mol Biol. 4:4-10, 1991.
37. Tanswell A K, Freeman B A. Pulmonary antioxidant enzyme maturation in the fetal and neonatal rats. 1. Developmental profiles. Pediatr Res 18:584-7, 1984.
38. Thannickal V J, Fanburg B L. Activation of an H2O2-generating NADH oxidase in human lung fibroblasts by transforming growth factor beta 1. J Biol Chem. 270:30334-8, 1995.
39. Truong S V, Monick M M, Yarovinsky T O, Powers L S, Nyunoya T, Hunninghake G W. Extracellular signal-regulated kinase activation delays hyperoxia-induced epithelial cell death in conditions of Akt downregulation. Am J Respir Cell Mol Biol. 31:611-8, 2004.
40. Wang X, Ryter S W, Dai C, Tang Z L, Watkins S C, Yin X M, Song R, Choi A M. Necrotic cell death in response to oxidant stress involves the activation of the apoptogenic caspase-8/bid pathway. J Biol Chem. 278:29184-91, 2003.
41. White C W, Avraham K B, Shanley P F, Groner Y. Transgenic mice with expression of elevated levels of copper-zinc superoxide dismutase in the lungs are resistant to pulmonary oxygen toxicity. J. Clin. Invest. 87:2162-68, 1991.
42. Wispe J R, Warner B B, Clark J C, Dey C R, Neuman J. Human Mn-superoxide dismutase in pulmonary epithelial cells of transgenic mice confers protection from oxygen injury. J. Biol. Chem. 267:23937-41, 1992.
43. Xu D, Finkel T. A role for mitochondria as potential regulators of cellular life span. Biochem Biophys Res Commun. 294:245-8, 2002.
44. Yoon M, Madden M C, Barton H A. Developmental expression of aldehyde dehydrogenase in rat: a comparison of liver and lung development. Toxicol Sci. 89:386-398, 2006.
45. Yunoya, T, Monick M M, Powers L S, Yarovinsky T O, Hunninghake G W. Macrophages survive hyperoxia via prolonged ERK activation due to phosphatase down-regulation. J Biol Chem. 280:26295-302, 2005.

We claim:

1. A plasmid containing at least one coding sequence for mitochondrial aldehyde dehydrogenase.

2. The plasmid of claim 1, said coding sequence being cloned into the plasmid vector pcDNA3.1.

3. The plasmid of claim 1, said coding sequence having a primer selected from the group consisting of SEQ ID NO. 1 and SEQ ID NO. 2.

4. The plasmid of claim 1, said coding sequence having at least 90% sequence homology with SEQ ID NO. 3.

5. The plasmid of claim 1, said coding sequence being human.

6. A cell transfected with the plasmid of claim 1.

7. The cell of claim 6, said cell expressing mitochondrial aldehyde dehydrogenase at a higher level than an untransfected cell in normoxic or hyperoxic conditions.

8. The cell of claim 6, said cell being from an epithelial tissue.

9. A method of ameliorating the effects of oxygen toxicity comprising the step of:
causing a cell to overexpress mitochondrial aldehyde dehydrogenase.

10. The method of claim 9, further comprising the step of transfecting said cell with a plasmid encoding for mitochondrial aldehyde dehydrogenase.

11. The method of claim 10, said transfected cell containing coding sequences having at least 90% sequence homology with SEQ ID NO. 3.

12. A method of ameliorating the effects of reactive oxygen species comprising the step of:
causing a cell to overexpress mitochondrial aldehyde dehydrogenase.

13. The method of claim 12, further comprising the step of transfecting said cell with a plasmid encoding for mitochondrial aldehyde dehydrogenase.

14. The method of claim 13, said transfected cell containing coding sequences having at least 90% sequence homology with SEQ ID NO. 3.

15. A method of activating a pathway selected from the group consisting of the ERK/MAPK pathway, the PI3K/Akt, and combinations thereof comprising the step of:
causing a cell to overexpress mitochondrial aldehyde dehydrogenase.

16. The method of claim 15, further comprising the step of transfecting said cell with a plasmid encoding for mitochondrial aldehyde dehydrogenase.

17. The method of claim 16, said transfected cell containing coding sequences having at least 90% sequence homology with SEQ ID NO. 3.

18. A method of reducing the incidence of, severity of, or likelihood of an individual developing chronic lung disease or bronchopulmonary dysplasia comprising the step of:
causing a cell to overexpress mitochondrial aldehyde dehydrogenase.

19. The method of claim 18, further comprising the step of transfecting said cell with a plasmid encoding for mitochondrial aldehyde dehydrogenase.

20. The method of claim 19, said transfected cell containing coding sequences having at least 90% sequence homology with SEQ ID NO. 3.


SEQUENCE LISTING (3 sequences)

SEQ ID NO: 1 - DNA, 24 bp, Homo sapiens
atgttgcgcgctgccgcccgcttc

SEQ ID NO: 2 - DNA, 21 bp, Homo sapiens
tgagttcttctgaggcacgac

SEQ ID NO: 3 - DNA, 1551 bp, Homo sapiens
atgttgcgcgctgccgcccgcttcgggccccgcctgggccgccgcctcttgtcagccgccgccacccaggccgtgcctgcccccaaccagcagcccgaggtcttctgcaaccagattttcataaacaatgaatggcacgatgccgtcagcaggaaaacattccccaccgtcaatccgtccactggagaggtcatctgtcaggtagctgaaggggacaaggaagatgtggacaaggcagtgaaggccgcccgggccgccttccagctgggctcaccttggcgccgcatggacgcatcacacaggggccggctgctgaaccgcctggccgatctgatcgagcgggaccggacctacctggcggccttggagaccctggacaatggcaagccctatgtcatctcctacctggtggatttggacatggtcctcaaatgtctccggtattatgccggctgggctgataagtaccacgggaaaaccatccccattgacggagacttcttcagctacacacgccatgaacctgtgggggtgtgcgggcagatcattccgtggaatttcccgctcctgatgcaagcatggaagctgggcccagccttggcaactggaaacgtggttgtgatgaaggtagctgagcagacacccctcaccgccctctatgtggccaacctgatcaaggaggctggctttccccctggtgtggtcaacattgtgcctggatttggccccacggctggggccgccattgcctcccatgaggatgtggacaaagtggcattcacaggctccactgagattggccgcgtaatccaggttgctgctgggagcagcaacctcaagagagtgaccttggagctgggggggaagagccccaacatcatcatgtcagatgccgatatggattgggccgtggaacaggcccacttcgccctgttcttcaaccagggccagtgctgctgtgccggctcccggaccttcgtgcaggaggacatctatgatgagtttgtggagcggagcgttgcccgggccaagtctcgggtggtcgggaacccctttgatagcaagaccgagcaggggccgcaggtggatgaaactcagtttaagaagatcctcggctacatcaacacggggaagcaagagggggcgaagctgctgtgtggtgggggcattgctgctgaccgtggttacttcatccagcccactgtgtttggagatgtgcaggatggcatgaccatcgccaaggaggagatcttcgggccagtgatgcagatcctgaagttcaagaccatagaggaggttgttgggagagccaacaattccacgtacgggctggccgcagctgtcttcacaaaggatttggacaaggccaattacctgtcccaggccctccaggcgggcactgtgtgggtcaactgctatgatgtgtttggagcccagtcaccctttggtggctacaagatgtcggggagtggccgggagttgggcgagtacgggctgcaggcatacactgaagtgaaaactgtcacagtcaaagtgcctcagaagaactca